(19) United States
(12) Patent Application Publication
(10) Pub. No.: US 2012/0143075 A1
(43) Pub. Date: Jun. 7, 2012

(54) Apparatus and method for monitoring, analyzing, and utilizing brainwave and biologic data at transition points along the neurochromometric sequence whereby a stimulus presented to the central nervous system results in cognition and volitional action

(76) Inventor: Michael Anselme Tansey, Hillsborough, NJ (US)

(21) Appl. No.: 13/373,769

(22) Filed: Nov. 30, 2011

Related U.S. Application Data
(60) Provisional application No. 61/459,085, filed on Dec. 7, 2010.

(51) Int. Cl. A61B 5/0476

(52) U.S. Cl. 600/544

(57) ABSTRACT

Apparatus and method for monitoring, analyzing, and utilizing brainwave and biologic data at transition points along the neurochromometric sequence whereby a stimulus is presented to the central nervous system results in cognition and volitional action.

### BACKGROUND OF THE INVENTION

1. Field of the Invention

As per Provisional Patent Application 61/459,085, this invention comprises an apparatus and method for monitoring, analyzing, and utilizing brainwave and biologic data at transition points along the neurochronometric sequence whereby a stimulus presented to the central nervous system results in cognition and volitional action

2. Descriptions of Related Art

Tansey, Michael A, Tachiki, K., & Tansey, J. A. 1994. Cartography of conscious States: A functional re-examination of theta, alpha, and beta. Subtle Energies, 4, p. 137. sets forth the commonly accepted view in the field of neuroscience wherein: “The brain sets the stage of behavior—micro-temporally. The requisite brain-biologic correlates for cognitive behavior are functionally matched and sorted according to an evolving cognitive-brain archetype—in fractions of a second . . . these micro-state neural nets, along with stimulus refinement and associative response, define and determine cognition. The reflections and correlates of “mind” are also to be found in the interleaved energies of the brain's orchestration of individual functional manifestation.” In simpler terms, brain activation networks in the cerebral cortex repeatedly change the state of coordination among their constituent areas on a sub-second time scale to ongoingly enable/inform one's conscious awareness and determines their cognitive state. As such, “Alterations of brain activation nets yield functional changes in the cognitive state and hence the state of mind of the individual” (Tansey, Michael A, Tachiki, K., & Tansey, J. A. 1994. Cartography of conscious States: A functional re-examination of theta, alpha, and beta. Subtle Energies, 4, p. 146). These pre-cognitive changes in the brainwave activation of regional neural nets can be recordable/observable via changes in their specific brainwave activity. The characteristics of the pre-cognitive aspects of one's biologic and brainwave energy signature is what, ultimately, informs/enables one's flow of consciousness and one's cognitive state.

Cognition is a term, which has traditionally been used to refer to one's first person awareness of one's own flow of indwelling consciousness awareness of self and mind. As such one's cognitive state is understood to include diverse mental processes—such as unverbalized and verbalized thinking, conceiving, perceiving, and reasoning, one's awareness of the truth and falsehood of information and self-verbalizations, and to also include any class of mental “behavior” involving symbolizing, insight, expectancy, complex rule usage, intentionality, problem solving and imagery. When one attends to one's cognitive state/flow of consciousness it is then amenable to internal acknowledgment, critique, self-assessment as to one's conjectural accuracy as well as the level of accuracy of one's truthfulness in conveying knowledge and that of data presented by others, and subsequent mental self-talk, prior to any outwardly observable physiological response to it. Digital analysis of the electrical properties of human brainwave activity (cycle-per-second waveforms) provides greater specificity, over the prior art, as to the functional correlates of the brain's pre-cognitive, cognitive, somatosensory, and higher order mental function—the electrophysiological substrate of one's flow of consciousness.

The pre-conscious, pre-cognitive, brain state is different from the conscious, self-aware cognitive state. Pre-cognitive brainwave activation changes enabling a resultant cognitive state are analyzable and identifiable prior to one's conscious awareness of their impact on one's flow of consciousness and cognitive state. As experimentally demonstrated and described by Libet (1990) pre-cognitive changes may take up to one-and-a-half seconds to manifest—without any awareness of such changes taking place by the indwelling consciousness of the individual (B. Libet, 1990, Cerebral Processes that Distinguish Conscious Experience from Unconscious Mental Function. In The Principles of Design and Operation of the Brain. J. C. Eckles & O. D. Creutzfeldr, Eds. Experimental Brain Research Series 21, Springer-Verlog, Berlin, pp. 185-205). Libet replicated the results of Deeke, Grotzinger and Kornhuber; (L. Deeke, B. Grotzinger & H. H. Kornhuber. Voluntary Finger Movements in Man: Cerebral Potentials and Theory. Biological Cybernetics, 23, pp. 99-106) wherein they quantified and isolated Electroencephalographic (EEG) brainwave changes enabling what was to be a spontaneous act of individual volition—flexing a finger. While the people in these studies consciously thought that they were instantly and spontaneously flexing their finger, their brains' were observed to be building up the electrical potentials (pre-cognitively) leading to the finger flex for from one second to one-and-a-half seconds prior to their avowed spontaneous conscious execution of the finger flex.

In monitoring, analyzing and utilizing brainwave and biologic data there are three key transition points along the path whereby a stimulus presented to the central nervous system results in cognition and volitional action: EEG Stage 1. The pre-stimulus state of activation immediately prior to stimulus introduction. EEG Stage 2. The instant of stimulus presentation which produces an automatic cerebral cascade of related neural net activation. EEG Stage 3. The the elapsed time between the presentation of a stimulus and the subsequent behavioral response—In neuropsychology it is considered to be an index of how fast the thinker can execute the mental operations needed by the task at hand.

Conventional EEG methods and apparatus reference EEG energy in wide bands. Wide band (Delta, Theta, Alpha, and Beta) EEG methodology first takes all of the energy obtained found within the 0.5 Hz to 1 Hz, the 1 Hz, the 2 Hz, the 3 Hz, and the 4 Hz brainwave bands, averages all of the summed energy found therein, calls this averaged energy output “Delta”, and then utilizes it as an independent stand alone measure of EEG activity. Then, the wide band EEG methodology takes all of the energy obtained from within the 4 Hz, the 5 Hz, the 6 Hz, the 7 Hz, and the 8 Hz brainwave bands, sums it up, averages all of the energy found therein by 5, calls it “Theta”, and uses it as an independent stand alone measure of EEG activity. Then, the wide band EEG methodology takes all of the energy obtained from within the 8 Hz, the 9 Hz, the 10 Hz, the 11 Hz, and the 12 Hz brainwave bands, averages all of the energy found therein and calls it “Alpha”, and uses it as an independent stand alone measure of EEG activity. Then, the wide band Q-EEG takes all of the energy obtained from within the 13 Hz, the 14 Hz, the 15 Hz, the 16 Hz, the 17 Hz, the 18 Hz, the 19 Hz, the 20 Hz, the 21 Hz, the 22 Hz, the 23 Hz, the 24 Hz and the 25 Hz brainwave bands, averages all of the energy found therein and calls it “Beta”. Additionally, wide band activity called Sensorimotor Rhythm (SMR) takes all of the energy found in the 12 Hz, the 13 Hz, the 14 Hz, and the 15 Hz brainwave bands, averages all of the energy found therein and calls it “SMR”, and uses it as an independent stand alone measure of EEG activity. Several patents have been directed to monitoring EEG in terms of the sensed amplitudes and percentages of alpha, theta, beta, delta, and SMR brainwave activity.

U.S. Pat. No. 4,928,704 describes a biofeedback method and system for training a person to develop useful degrees of voluntary, control of personal EEG activity. EEG sensors are attached to cortical sites on a head of a person for sensing EEG energy. EEG electrical energy is filtered into the pre-defined sub-bands of alpha, theta, beta, and delta. Other patents directed to EEG biofeedback with alpha, theta, beta, delta, and SMR brainwave bands include U.S. Pat. Nos. 3,855,988: 4,140,997; 4,883,067; 4,919,143; 5,024,235, and European Patent No. 367,106.

U.S. Pat. No. 4,746,751 describes a system for displaying multichannel EEG data. In performing this, the procedure and method entails Evoked Response Potential signal averaging (ERP). A summed signal averaged brain map may be pieced together being comprised of reflections of the average amount of overall energy monitored over many electrode sites. In ERP, the subject receives a set of stimuli, which evoke brainwaves. Other examples of patents directed to ERP include U.S. Pat. Nos. 4,498,080; 4,926,969, 5,564,433 and PCT Patent Application No. 8303745.

A Major disadvantage to conventional wide band (Delta, Theta, Alpha, and Beta) EEG biofeedback and ERP studies has been poor resolution of brainwave bands produced by conventional bandpass filters used as a front end for signal processing electronics. Another drawback has been that the bandpass filters are easily overloaded by an upsurge of electrical activity or via high amplitude slow waves. An upsurge in electrical activity accompanies muscle movement and high amplitude slow wave activity accompanies many cerebral disorders. These unwanted, and all to frequent, signal contaminating sources are referred to as artifacts. Bandpass analysis is dependent on differential amplifiers, which multiply many thousands of times (i.e., as much as 50,000 times) biologic signals and accompanying artifacts. Such electronic/signal refiners contribute to inaccuracy in the monitoring of the EEG signals—beyond the limitations of being limited to delta, theta, alpha, beta and SMR representations.

Another disadvantage is conventional mechanical bandpass filtering is their use of arbitrary and inexact bandwidths in defining specific brain states. A more exact analysis of electroencephalographic waveforms is essential to EEG analysis protocols. Additional drawbacks to conventional systems are their reliance on multiplexors, which may sample the bandpass, configured signal at a rate of one sampling per second per waveform. The prior art also has the disadvantage of being limited to pre-defining their sampling of EEG data into delta, theta, alpha, beta, and SMR representations. Spectral analysis and Fourier Transforms have been noted to encode our experience of our surrounding environment.

The prior art is lacking in usage of Spectral Digital analysis and Fourier Transforms in resolving their EEG data. This invention does use Spectral Analysis and Fourier Transforms to as accurately as possible resolve EEG data with as much fidelity as possible.

U.S. Pat. No. 5,406,957 describes a system for simultaneously monitoring and manipulating brainwaves in a continuum from 0 to 90 Hz; claiming an apparatus for monitoring a brainwave signature of a cognitive state of a person.

An apparatus and method for monitoring, analyzing, and utilizing brainwave and biologic data at transition points along the neurochronometric path whereby a stimulus presented to the central nervous system results in cognition and volitional action is not in the prior art. In addition, analysis of same biologic and EEG brainwave data using Spectral Analysis and Fourier Transforms in a continuous stream—from 0 to 150 Hz, is not in the prior art. Resolving the same bioelectric and EEG brainwave signals in a continuous spectrum in the range of about 0 to about 150 Hertz with resolution of bandwidths of one hertz or less around a given frequency—is not in the prior art.

### BRIEF DESCRIPTION OF THE DRAWINGS

FIG. 1 is a schematic diagram of the system in accordance with the present invention.

### SUMMARY OF THE INVENTION

This invention comprises an apparatus and method for monitoring, analyzing, and utilizing brainwave and biologic data at transition points along the neurochronometric path whereby a stimulus presented to the central nervous system results in cognition and volitional action. In this, this invention is oriented towards monitoring, analyzing and utilizing brainwave and biologic data in three key transition points along the path whereby a stimulus presented to the central nervous system results in cognition and volitional action: EEG Stage 1. The pre-stimulus state of activation immediately prior to stimulus introduction. EEG Stage 2. The instant of stimulus presentation producing an automatic cerebral cascade of related neural net activation. EEG Stage 3. The elapsed time between the presentation of a stimulus and the subsequent behavioral response—In neuropsychology it is considered to be an index of how fast the thinker can execute the mental operations needed by the task at hand.

In a preferred embodiment, an active electrode is mounted along the midline of the skull of the person. A reference electrode and a ground electrode are placed on opposite ears of the person. Bioelectric and EEG signals of the person are detected by the electrode array which may also be expanded to utilize all of the 10/20 system EEG electrode placement sites in accord with the teachings of the present invention.

A preamplifier amplifies and optically isolates the detected bioelectric and EEG brainwave signals. The amplified data is transmitted to a signal processor. Preferably a digital signal processor installed in a computer. A fast Fourier transform is performed on the bioelectric and EEG brainwave signals. The bioelectric and EEG brainwave signals will be in the 0 to about 150 Hz; with resolution of bandwidths of one hertz or less around a given frequency. Raw bioelectric and EEG brainwave signals and subsequent analysis may be stored in the memory of the computer or by other means.

### DETAILED DESCRIPTION OF THE INVENTION

During the course of this description like numbers will be used to identify the elements according to the different figures, which illustrate the invention.

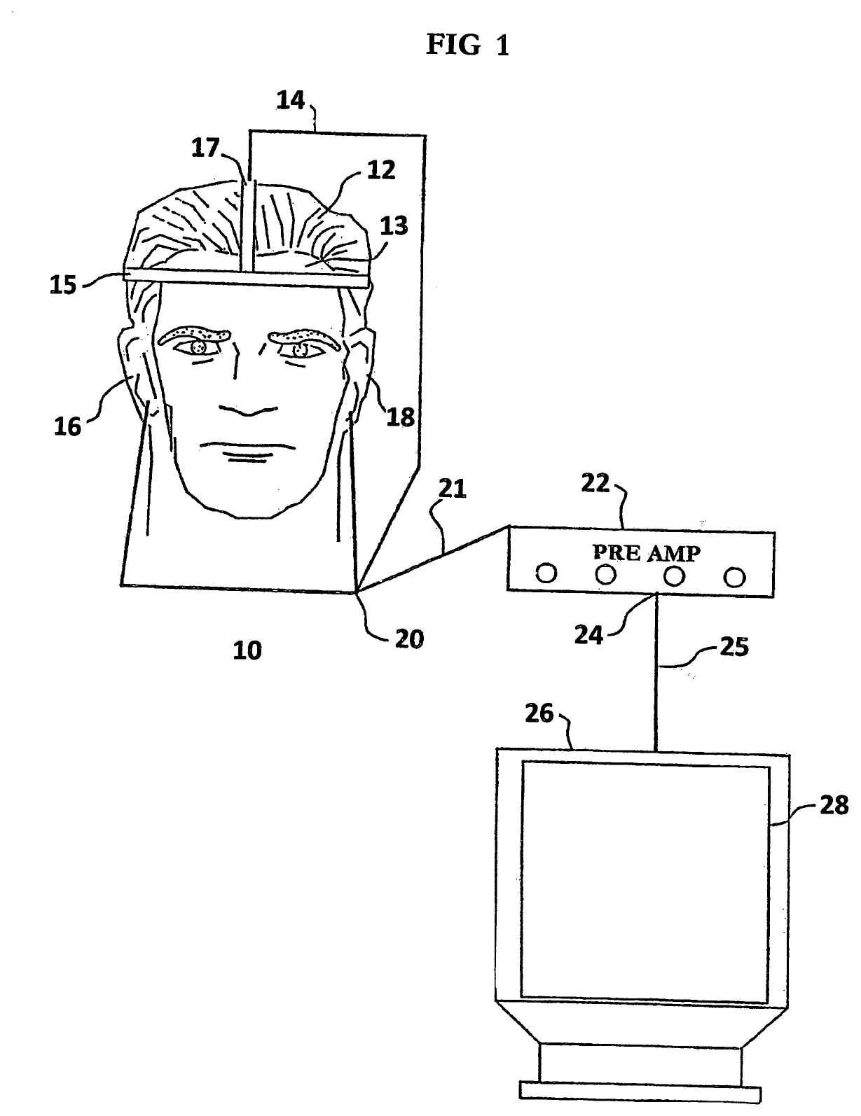

FIG. 1. Is a schematic diagram of an apparatus and method for monitoring, analyzing and utilizing brainwave and biologic data characterizing a pre-cognitive state, in accordance with the present invention. In a preferred embodiment, a person 12 is monitored with three electrodes. Using three electrodes, active electrode 14 is mounted as comfortably as possible along the midline of the skull of person 12. Reference electrode 16 is placed on one ear of person 12 and ground electrode 18 is placed on the other ear of person 12. Preferably reference electrode 16 and ground electrode 18 are attached with ear clips. It will be appreciated by those skilled in the art that various sizes, shapes, and configurations of multiple electrodes (such as utilizing the 10/20 system of electrode placement) can be used with the teachings of the present invention.

In the three-electrode configuration, the active electrode 14 is preferably placed along the midline of the top of the skull to overlay overlay the cerebral longitudinal fissure of person 12. An elasticized headband 15 is placed around the head parallel to the eyebrows and across the middle of the forehead 13 of person 12. A second elasticized band 17 is placed across the top of the head of person 12 and attaches to first band 15. It will be appreciated by those skilled in the art that various sizes, shapes, and configurations of multiple electrodes (such as utilizing the 10/20 system of electrode placement) can be used with the teachings of the present invention. In the alternative, active electrode 14 and ground electrode 18, and reference electrode 16 may be incorporated into a skullcap utilizing the 10/20-electrode placement system.

Bioelectric signal 20 detected by the electrodes placed about the skull of person 12, are conveyed by line 21 to the preamplifier 22. It will be appreciated by those skilled in the art that conveyance of the bioelectric signal 20 may be via wireless or infrared remote transmission instead of via a hard wire connection to the preamplifier 22. Bioelectric signals 20 can be in the range of 0 to about 150 Hz; with resolution of bandwidths of one hertz or less around a given frequency.

The remote transmitter can be attached to first headband 15. The remote transmitter has the advantage of allowing free movement of person 12 during the monitoring of bioelectric signal 20. Preamplifier 22 amplifies and optically isolates the bioelectric signal 20. Amplified bioelectric signal 24 is applied by hard line 25 to the signal analysis component of the computer 26. A Fast Fourier transform (FFT), and other bioelectric signal analyses are performed via software within the computer 26 and displayed as desired on the computer screen 28 or stored within the computer or other external data storage device. It will be appreciated by those skilled in the art that conveyance of the bioelectric signal 20 may be transmitted to a pre-amplifier capability built into computer 26 rather than using separate preamplifier 22.

The present invention has the advantage of monitoring bioelectric signals in a continuous spectrum in the range of about 0 to about 150 Hertz with resolution of bandwidths of one hertz or less around a given frequency. While the invention has been described with reference to the preferred embodiment, this description is not intended to be limiting. It will be appreciated by those of ordinary skill in the art that modifications may be made without departing from the spirit and scope of the invention.

I claim:

1. An apparatus and method for monitoring, analyzing, and utilizing brainwave and biologic data along the neurochronometric sequence whereby a stimulus presented to the central nervous system results in cognition and volitional action. The bioelectric and EEG brainwave signals will be evaluated in a continuous spectrum in the range of about 0 to about 150 Hertz with resolution of bandwidths of one hertz or less around a given frequency.

2. The apparatus and method of claim 1 further comprising monitoring and analyzing means whereby EEG and biologic data are additionally measured at three key transition points along the path whereby a stimulus presented to the central nervous system results in cognition and volitional action: EEG Stage 1. The pre-stimulus state of activation immediately prior to stimulus introduction to the central nervous system. EEG Stage 2. The instant of stimulus presentation producing an automatic cerebral cascade of related neural net activation. EEG Stage 3. The elapsed time between the presentation of a stimulus and the subsequent behavioral response. In neuropsychology it is considered to be an index of how fast the thinker can execute the mental operations needed by the task at hand. The bioelectric and EEG brainwave signals will be evaluated in a continuous spectrum in the range of about 0 to about 150 Hertz with resolution of bandwidths of one hertz or less around a given frequency.

3. The apparatus of claim 1 further comprising signal processing means, said processing means including a preamplifier which amplifies and optically isolates the detected bioelectric and EEG brainwave signals. The amplified data is transmitted to a digital signal processor. A Fast Fourier transform is performed on the bioelectric and EEG brainwave signals. The bioelectric and EEG brainwave will be evaluated in a continuous spectrum in the range of about 0 to about 150 Hertz with resolution of bandwidths of one hertz or less around a given frequency. Raw and manipulated bioelectric and EEG brainwave signals and subsequent analysis may be stored in the memory of the computer or by other means.

4. The apparatus and method of claim 1 further comprising monitoring and analyzing means whereby EEG and biologic data are additionally measured to contrast EEG Stage 1, from EEG Stage 2, From EEG Stage 3. In this the unconscious and/or pre-cognitive state will be differentiated from the conscious and/or cognitive state of a person's central nervous system responding to a stimulus or outside influence.
